(19) United States
(12) Patent Application Publication
(10) Pub. No.: US 2006/0052676 A1
(43) Pub. Date: Mar. 9, 2006

(54) Tele-presence system that allows for remote monitoring/observation and review of a patient and their medical records

(76) Inventors: Yulun Wang, Goleta, CA (US); Charles S. Jordan, Santa Barbara, CA (US); Jonathan Southard, Santa Barbara, CA (US); Marco Pinter, Santa Barbara, CA (US)

Correspondence Address: IRELL & MANELLA LLP, 840 NEWPORT CENTER DRIVE, SUITE 400, NEWPORT BEACH, CA, 92660, US

(21) Appl. No.: 10/936,041

(22) Filed: Sep. 7, 2004

(51) Int. Cl. A61B 5/00

(52) U.S. Cl. 600/300

(57) ABSTRACT

A system that includes a mobile platform and a remote station. The remote station may be a personal computer coupled to the remote platform through a broadband network. A user can control movement of the mobile platform through the remote station. A medical monitoring device such as a stethoscope or EKG monitor can be coupled to the mobile platform and used to take patient data. The data can be transmitted to the remote station by the mobile platform. The medical monitoring device(s) may be wirelessly coupled to the mobile platform. The system may include a server that can provide an electronic medical record to the remote station. The remote station may have a monitor that displays the electronic medical record and an image captured by a camera of the mobile platform. The system allows a doctor at the remote station to more fully examine a patient while viewing past medical records.

### BACKGROUND OF THE INVENTION

1. Field of the Invention

The subject matter disclosed generally relates to the field of two-way teleconferencing.

2. Background Information

There is a growing need to provide remote health care to patients that have a variety of ailments ranging from Alzheimers to stress disorders. To minimize costs it is desirable to provide home care for such patients. Home care typically requires a periodic visit by a health care provider such as a nurse or some type of assistant. Due to financial and/or staffing issues the health care provider may not be there when the patient needs some type of assistance. Additionally, existing staff must be continuously trained, which can create a burden on training personnel. It would be desirable to provide a system that would allow a health care provider to remotely care for a patient without being physically present.

Robots have been used in a variety of applications ranging from remote control of hazardous material to assisting in the performance of surgery. For example, U.S. Pat. No. 5,762,458 issued to Wang et al. discloses a system that allows a surgeon to perform minimally invasive medical procedures through the use of robotically controlled instruments. One of the robotic arms in the Wang system moves an endoscope that has a camera. The camera allows a surgeon to view a surgical area of a patient.

Tele-robots such as hazardous waste handlers and bomb detectors may contain a camera that allows the operator to view the remote site. Canadian Pat. No. 2289697 issued to Treviranus, et al. discloses a teleconferencing platform that has both a camera and a monitor. The platform includes mechanisms to both pivot and raise the camera and monitor. The Treviranus patent also discloses embodiments with a mobile platform, and different mechanisms to move the camera and the monitor.

U.S. Pat. Application Pub. No. U.S. 2001/0054071 filed in the name of Loeb, discloses a video-conferencing system that includes a number of graphical user interfaces (“GUIs”) that can be used to establish a video-conference. One of the GUIs has an icon that can be selected to make a call. The Loeb application discloses stationary video-conferencing equipment such as a television. There is no discussion in Loeb about the use of robotics.

There has been marketed a mobile robot introduced by InTouch-Health, Inc., the assignee of this application, under the trademarks COMPANION and RP-6. The InTouch robot is controlled by a user at a remote station. The remote station may be a personal computer with a joystick that allows the user to remotely control the movement of the robot. Both the robot and remote station have cameras, monitors, speakers and microphones to allow for two-way video/audio communication.

It would be desirable to upgrade the InTouch system to perform more functions than remotely viewing and communicating with a patient. It would be desirable if the robot could actually obtain monitoring data such as heart rate, EKG, etc. It would also be desirable to provide a remote controlled robotic system with an ability to call up electronic medical records so that the doctor can review the records while examining the patient through the robot.

### BRIEF SUMMARY OF THE INVENTION

A robot system that includes a remote station that is coupled to a mobile platform. The system may include a medical monitoring device that is coupled to the mobile platform. The remote station may have a monitor that displays an image captured by a camera of the mobile platform and an electronic medical record.

### BRIEF DESCRIPTION OF THE DRAWINGS

FIG. 1 is an illustration of a robotic system;

FIG. 2 is a schematic of an electrical system of a robot;

FIG. 3 is a further schematic of the electrical system of the robot;

FIG. 4 is a display user interface of a remote station;

FIG. 5 is a display user interface showing an electronic medical record;

FIG. 6 is a display user interface showing an image and an electronic medical record being simultaneously displayed.

### DETAILED DESCRIPTION

Disclosed is a system that includes a mobile platform and a remote station. The remote station may be a personal computer coupled to the remote platform through a broadband network. A user can control movement of the mobile platform through the remote station. A medical monitoring device such as a stethoscope or EKG monitor can be coupled to the mobile platform and used to take patient data. The data can be transmitted to the remote station by the mobile platform. The medical monitoring device(s) may be wirelessly coupled to the mobile platform. The system may include a server that can provide an electronic medical record to the remote station. The remote station may have a monitor that displays the electronic medical record and an image captured by a camera of the mobile platform. The system allows a doctor at the remote station to more fully examine a patient while viewing past medical records.

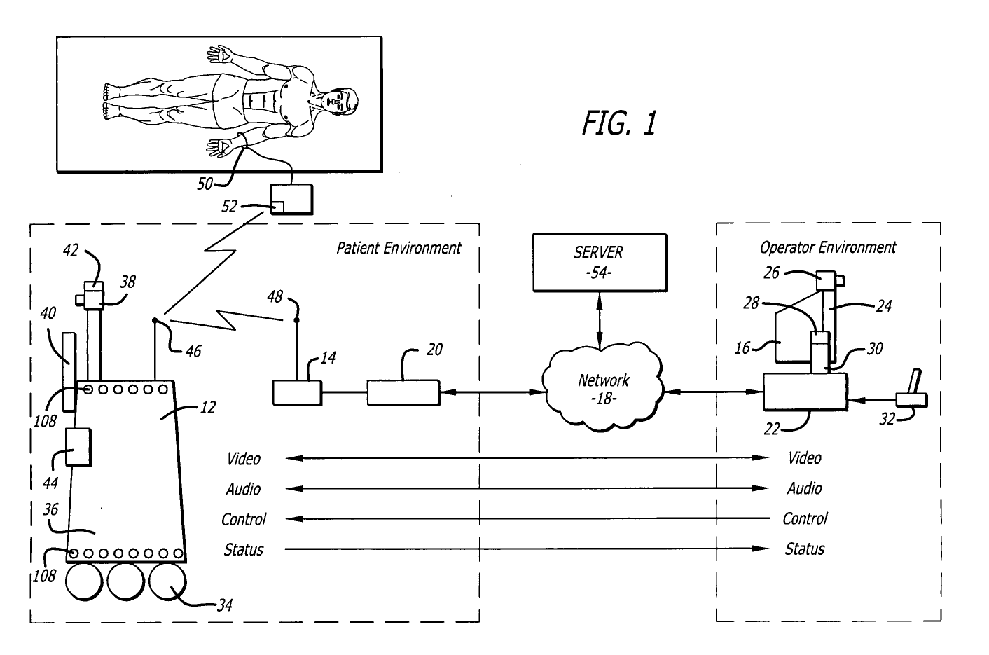

Referring to the drawings more particularly by reference numbers, FIG. 1 shows a system 10. The robotic 10 includes a robot 12, a base station 14 and a remote control station 16. The remote control station 16 may be coupled to the base station 14 through a network 18. By way of example, the network 18 may be either a packet switched network such as the Internet, or a circuit switched network such has a Public Switched Telephone Network (PSTN) or other broadband system. The base station 14 may be coupled to the network 18 by a modem 20 or other broadband network interface device. By way of example, the base station 14 may be a wireless router. Alternatively, the robot 12 may have a direct connection to the network thru for example a satellite.

The remote control station 16 may include a computer 22 that has a monitor 24, a camera 26, a microphone 28 and a speaker 30. The computer 22 may also contain an input device 32 such as a joystick or a mouse. The control station 16 is typically located in a place that is remote from the robot 12. Although only one remote control station 16 is shown, the system 10 may include a plurality of remote stations. In general any number of robots 12 may be controlled by any number of remote stations 16 or other robots 12. For example, one remote station 16 may be coupled to a plurality of robots 12, or one robot 12 may be coupled to a plurality of remote stations 16, or a plurality of robots 12.

Each robot 12 includes a movement platform 34 that is attached to a robot housing 36. Also attached to the robot housing 36 are a camera 38, a monitor 40, a microphone(s) 42 and a speaker(s) 44. The microphone 42 and speaker 30 may create a stereophonic sound. The robot 12 may also have an antenna 46 that is wirelessly coupled to an antenna 48 of the base station 14. The system 10 allows a user at the remote control station 16 to move the robot 12 through operation of the input device 32. The robot camera 38 is coupled to the remote monitor 24 so that a user at the remote station 16 can view a patient. Likewise, the robot monitor 40 is coupled to the remote camera 26 so that the patient may view the user. The microphones 28 and 42, and speakers 30 and 44, allow for audible communication between the patient and the user.

The remote station computer 22 may operate Microsoft OS software and WINDOWS XP or other operating systems such as LINUX. The remote computer 22 may also operate a video driver, a camera driver, an audio driver and a joystick driver. The video images may be transmitted and received with compression software such as MPEG CODEC.

The robot 12 may be coupled to one or more medical monitoring devices 50. The medical monitoring device 50 can take medical data from a patient. By of example, the medical monitoring device 50 may be a stethoscope, a pulse oximeter and/or an EKG monitor. The medical monitoring device 50 may contain a wireless transmitter 52 that transmits the patient data to the robot 12. The wirelessly transmitted data may be received by antennae 46, or a separate antennae (not shown). The robot 12 can then transmit the data to the remote station 16.

The wireless transmission from the medical monitoring device 50 may be in accord with various wireless standards such as IEEE. The standard used to transmit data from the medical monitoring device 50 should not interfere with the wireless communication between the robot 12 and the base station 14. Although wireless transmission is shown and described, it is to be understood that the medical monitoring device 50 can be coupled to the robot 12 by wires (not shown).

The remote station 16 may be coupled to a server 54 through the network 18. The server 54 may contain electronic medical records of a patient. By way of example, the electronic medical records may include written records of treatment, patient history, medication information, x-rays, EKGs, laboratory results, physician notes, etc. The medical records can be retrieved from the server 54 and displayed by the monitor 24 of the remote station. In lieu of, or in addition to, the medical records can be stored in the mobile robot 12. The remote station 16 may allow the physician to modify the records and then store the modified records back in the server 54 and/or robot 12.

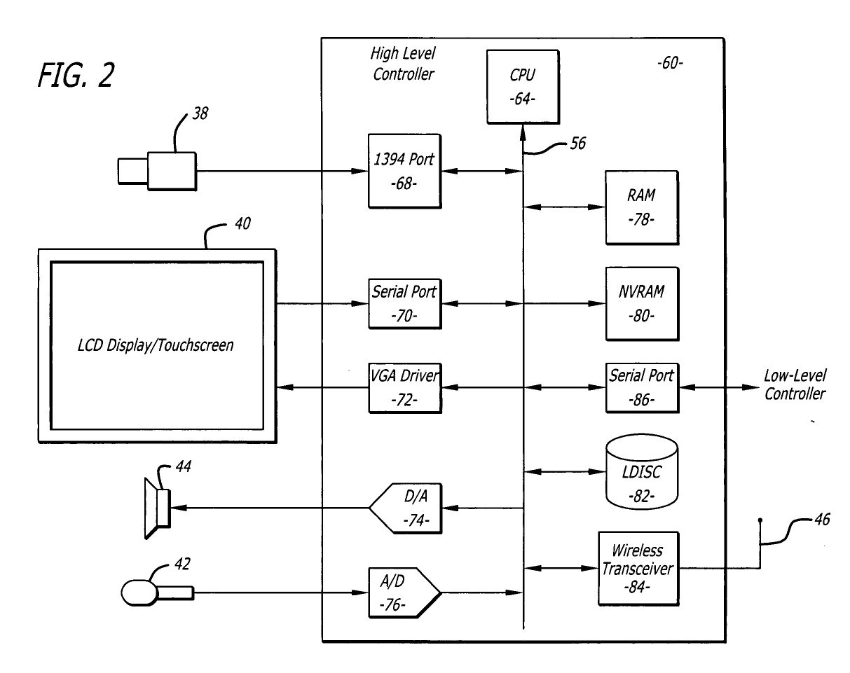

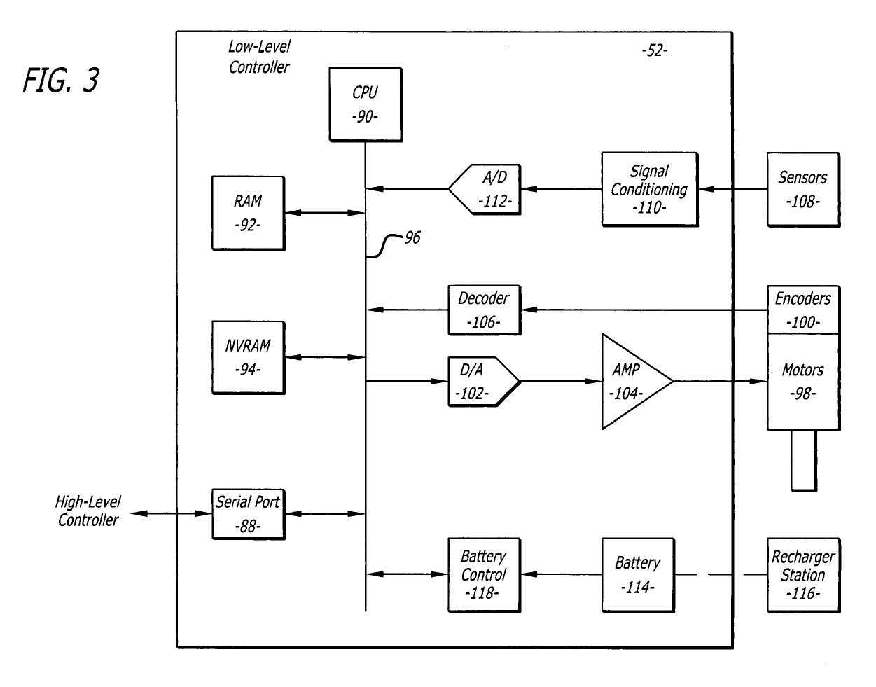

FIGS. 2 and 3 show an embodiment of a robot 12. Each robot 12 may include a high level control system 60 and a low level control system 62. The high level control system 60 may include a processor 64 that is connected to a bus 66. The bus is coupled to the camera 38 by an input/output (I/O) port 68, and to the monitor 40 by a serial output port 70 and a VGA driver 72. The monitor 40 may include a touchscreen function that allows the patient to enter input by touching the monitor screen.

The speaker 44 is coupled to the bus 66 by a digital to analog converter 74. The microphone 42 is coupled to the bus 66 by an analog to digital converter 76. The high level controller 60 may also contain random access memory (RAM) device 78, a non-volatile RAM device 80 and a mass storage device 82 that are all coupled to the bus 72. The mass storage device 82 may contain medical files of the patient that can be accessed by the user at the remote control station 16. For example, the mass storage device 82 may contain a picture of the patient. The user, particularly a health care provider, can recall the old picture and make a side by side comparison on the monitor 24 with a present video image of the patient provided by the camera 38. The robot antennae 46 may be coupled to a wireless transceiver 84. By way of example, the transceiver 84 may transmit and receive information in accordance with IEEE 802.11b. The transceiver 84 may also process signals from the medical monitoring device in accordance with IEEE also known as Bluetooth. The robot my have a separate antennae to receive the wireless signals from the medical monitoring device.

The controller 64 may operate with a LINUX OS operating system. The controller 64 may also operate MS WINDOWS along with video, camera and audio drivers for communication with the remote control station 16. Video information may be transceived using MPEG CODEC compression techniques. The software may allow the user to send e-mail to the patient and vice versa, or allow the patient to access the Internet. In general the high level controller 60 operates to control communication between the robot 12 and the remote control station 16.

The high level controller 60 may be linked to the low level controller 62 by serial ports 86 and 88. The low level controller 62 includes a processor 90 that is coupled to a RAM device 92 and non-volatile RAM device 94 by a bus 96. Each robot 12 contains a plurality of motors 98 and motor encoders 100. The motors 98 can activate the movement platform and move other parts of the robot such as the monitor and camera. The encoders 100 provide feedback information regarding the output of the motors 98. The motors 98 can be coupled to the bus 96 by a digital to analog converter 102 and a driver amplifier 104. The encoders 100 can be coupled to the bus 96 by a decoder 106. Each robot 12 also has a number of proximity sensors 108 (see also FIG. 1). The position sensors 108 can be coupled to the bus 96 by a signal conditioning circuit 110 and an analog to digital converter 112.

The low level controller 62 runs software routines that mechanically actuate the robot 12. For example, the low level controller 62 provides instructions to actuate the movement platform to move the robot 12. The low level controller 62 may receive movement instructions from the high level controller 60. The movement instructions may be received as movement commands from the remote control station or another robot. Although two controllers are shown, it is to be understood that each robot 12 may have one controller, or more than two controllers, controlling the high and low level functions.

The various electrical devices of each robot 12 may be powered by a battery(ies) 114. The battery 114 may be recharged by a battery recharger station 116. The low level controller 62 may include a battery control circuit 118 that senses the power level of the battery 114. The low level controller 62 can sense when the power falls below a threshold and then send a message to the high level controller 60.

The system may be the same or similar to a robotic system provided by the assignee InTouch-Health, Inc. of Santa Barbara, Calif. under the name RP-6, which is hereby incorporated by reference. The system may also be the same or similar to the system disclosed in application Ser. No. 10/206,457 published on Jan. 29, 2004, which is hereby incorporated by reference.

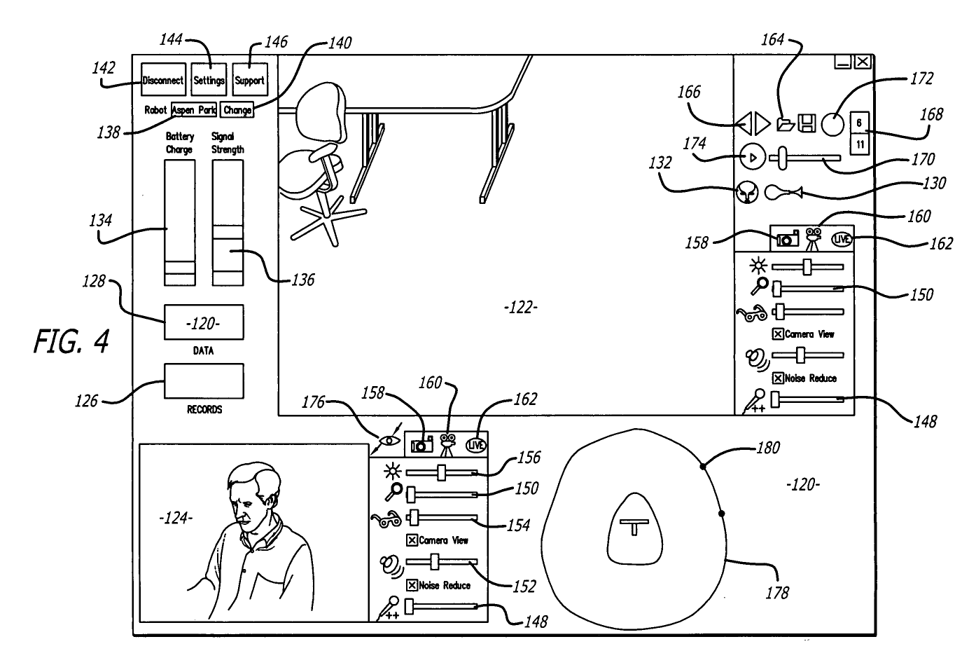

FIG. 4 shows a display user interface (“DUI”) 120 that can be displayed at the remote station 16 and/or the robot 12. The DUI 120 may include a robot view field 122 that displays a video image captured by the camera of the robot. The DUI 120 may also include a station view field 124 that displays a video image provided by the camera of the remote station 16. The DUI 120 may be part of an application program stored and operated by the computer 22 of the remote station 16.

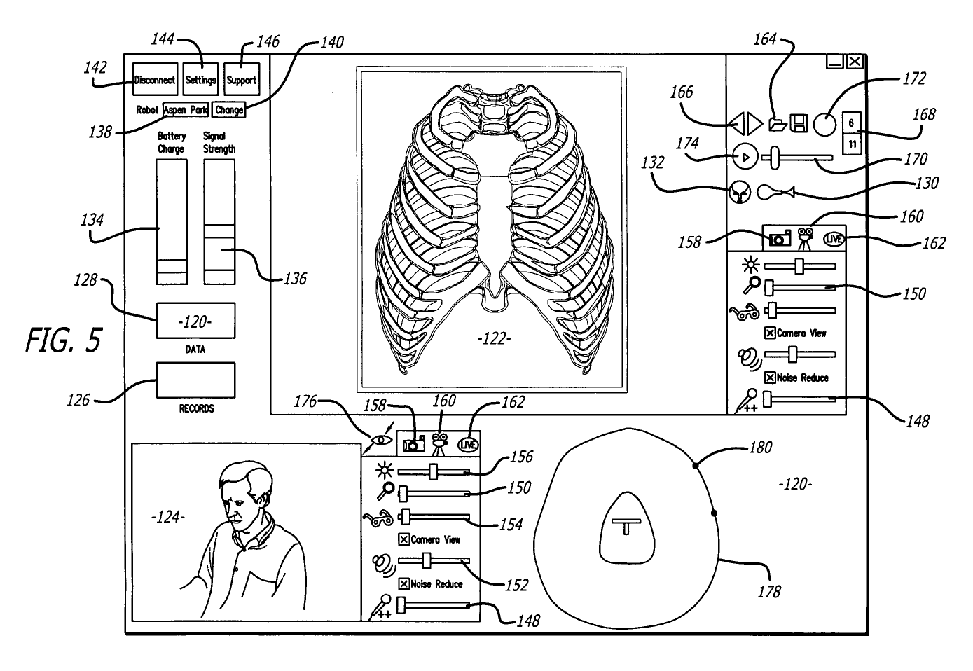

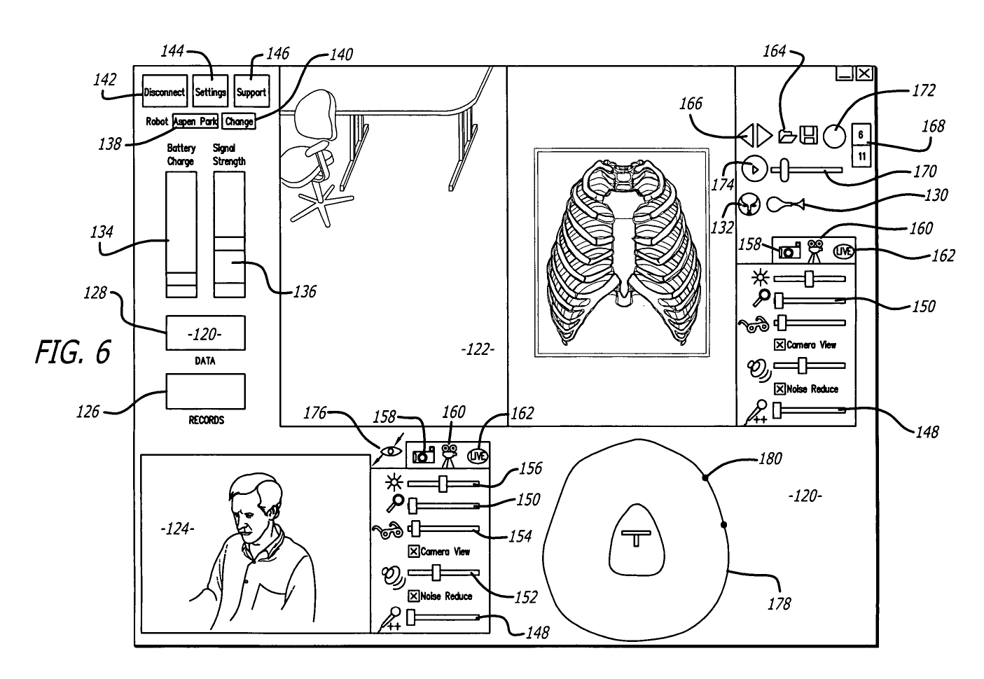

The DUI 120 may include a graphic button 126 that can be selected to display an electronic medical record as shown in FIG. 5. The button 126 can be toggled to sequentially view the video image and the electronic medical record. Alternatively, the view field 124 may be split to simultaneously display both the video image and the electronic medical record as shown in FIG. 6. The viewing field may allow the physician to modify the medical record by adding, changing or deleting all or part of the record. The remote clinician can also add to the medical record still images or video captured by the camera of the robot.

The DUI 120 may have a monitor data field 128 that can display the data generated by the medical monitoring device(s) and transmitted to the remote station. The data can be added to the electronic medical record, either automatically or through user input. For example, the data can be added to a record by “dragging” a monitor data field 128 into the viewing field 124.

The DUI 120 may include alert input icons 130 and 132. Alert icon 130 can be selected by the user at the remote station to generate an alert indicator such as a sound from the speaker of the robot. Selection of the icon generates an alert input to the robot. The robot generates a sound through its speaker in response to the alert input. By way of example, the sound may simulate the noise of a horn. Consequently, the icon may have the appearance of a horn. The remote station user may select the horn shaped icon 130 while remotely moving the robot to alert persons to the presence of the moving robot.

Alert icon 132 can be selected to request access to the video images from the robot. The default state of the robot may be to not send video information to the remote station. Selecting the alert icon 132 sends an alert input such as an access request to the robot. The robot then generates an alert indicator. The alert indicator can be a sound generated by the robot speaker, and/or a visual prompt on the robot monitor. By way of example, the visual prompt may be a “flashing” graphical icon. The sound may simulate the knocking of a door. Consequently, the alert icon 128 may have the appearance of a door knocker.

In response to the alert indicator the user may provide a user input such as the depression of a button on the robot, or the selection of a graphical image on the robot monitor, to allow access to the robot camera. The robot may also have a voice recognition system that allows the user to grant access with a voice command. The user input causes the robot to begin transmitting video images from the robot camera to the remote station that requested access to the robot. A voice communication may be established before the cycle of the alert input and response, to allow the user at the remote station to talk to the caller recipient at the robot.

The DUI 120 may include a graphical “battery meter” 134 that indicates the amount of energy left in the robot battery. A graphical “signal strength meter” 136 may indicate the strength of the wireless signal transmitted between the robot and the base station (see FIG. 1).

The DUI 120 may include a location display 138 that provides the location of the robot. The CHANGE button 140 can be selected to change the default robot in a new session. The user can initiate and terminate a session by selecting box 142. The box 142 changes from CONNECT to DISCONNECT when the user selects the box to initiate a session. System settings and support can be selected through buttons 144 and 146.

Both the robot view field 122 and the station view field 124 may have associated graphics to vary the video and audio displays. Each field may have an associated graphical audio slide bar 148 to vary the audio level of the microphone and another slide bar 150 to vary the volume of the speakers.

The DUI 120 may have slide bars 152, 154 and 156 to vary the zoom, focus and brightness of the cameras, respectively. A still picture may be taken at either the robot or remote station by selecting one of the graphical camera icons 158. The still picture may be the image presented at the corresponding field 122 or 124 at the time the camera icon 158 is selected. Capturing and playing back video can be taken through graphical icons 160. A return to real time video can be resumed, after the taking of a still picture, captured video, or reviewing a slide show, by selecting a graphical LIVE button 162.

A still picture can be loaded from disk for viewing through selection of icon 164. Stored still images can be reviewed by selecting buttons 166. The number of the image displayed relative to the total number of images is shown by graphical boxes 168. The user can rapidly move through the still images in a slide show fashion or move through a captured video clip by moving the slide bar 170. A captured video image can be paused through the selection of circle 172. Play can be resumed through button 174. Video or still images may be transferred to the robot by selecting icon 176.

A graphical depiction of the base of the robot can be shown in sensor field 178. The sensor may have various sensors that sense contact with another object. The sensor field 178 can provide a visual display of the sensors that detect the object. By way of example, the field may have one or more graphical dots 180 that display where on the robot the sensors detected an object. This provides the user with a sense of the robot environment that is outside the view of the robot camera.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

For example, although a robot is shown and described, it is to be understood that the system may contain any type of mobile platform that can take monitor data and/or capture images with a camera. The mobile platform may include a cart with a robot head that contains a camera, monitor, microphone and speakers. Additionally, the remote station may have a plurality of monitors for displaying various medical records, captured images, etc.

What is claimed is:

1. A system, comprising:
a mobile platform;
a medical monitoring device coupled to said mobile platform; and,
a remote station that is coupled to said mobile platform.

2. The system of claim 1, wherein said medical monitoring device is wireless coupled to said mobile platform.

3. The system of claim 1, wherein said medical monitoring device is a stethoscope.

4. The system of claim 1, wherein said medical monitoring device is a pulse oximeter.

5. The system of claim 1, wherein said medical monitoring device is an EKG monitor.

6. The system of claim 1, wherein said medical monitoring device transmits data to said mobile platform and said mobile platform transmits the data to said remote station.

7. The system of claim 1, wherein said mobile platform includes a camera and said remote station includes a monitor that displays an image provided by said camera.

8. The system of claim 7, wherein said remote station monitor displays an electronic medical record.

9. The system of claim 8, wherein said remote station monitor includes a display user interface that simultaneously displays the image and the electronic medical record.

10. The system of claim 8, further comprising a server that is coupled to said remote station and stores the electronic medical record.

11. A system, comprising:
a broadband network;
a mobile platform coupled to said broadband network;
a medical monitoring device coupled to said mobile platform; and,
a remote station that is coupled to said mobile platform through said broadband network.

12. The system of claim 11, wherein said medical monitoring device is wireless coupled to said mobile platform.

13. The system of claim 11, wherein said medical monitoring device is a stethoscope.

14. The system of claim 11, wherein said medical monitoring device is a pulse oximeter.

15. The system of claim 11, wherein said medical monitoring device is an EKG monitor.

16. The system of claim 11, wherein said medical monitoring device transmits data to said mobile platform, and said mobile platform transmits the data to said remote station through said broadband network.

17. The system of claim 11, wherein said mobile platform includes a camera and said remote station includes a monitor that displays an image provided by said camera.

18. The system of claim 17, wherein said remote station monitor displays an electronic medical record.

19. The system of claim 18, wherein said remote station monitor includes a display user interface that simultaneously displays the image and the electronic medical record.

20. The system of claim 18, further comprising a server that is coupled to said remote station through said broadband network and stores the electronic medical record.

21. A method for receiving medical monitoring data, comprising:
moving a mobile platform;
transmitting data from a medical monitoring device to the mobile platform; and,
transmitting the data from the mobile platform to a remote station.

22. The method of claim 21, wherein the data is wirelessly transmitted from the medical monitoring device to the mobile platform.

23. The method of claim 21, wherein the mobile platform moves in response to commands from the remote station.

24. The method of claim 23, wherein the data is transmitted from the mobile platform to the remote station through a broadband network.

25. The method of claim 21, further comprising capturing an image with a camera of the mobile platform, transmitting the image to the remote station and displaying the image on a monitor of the remote station.

26. The method of claim 25, further comprising displaying an electronic medical record on the monitor.

27. The method of claim 26, wherein the image and the electronic medical record are displayed simultaneously.

28. The method of claim 26, wherein the electronic medical record is received from a server.

29. A robot system, comprising:
a mobile platform with a camera that captures an image; and,
a remote station that is coupled to said mobile platform, said remote station includes a monitor that displays the image, said monitor can also display en electronic medical record.

30. The system of claim 29, wherein said monitor simultaneously displays the image and the electronic medical record.

31. The system of claim 29, wherein the electronic medical record is stored within said mobile platform.

32. The system of claim 29, further comprising a server that is coupled to said remote station and stores the electronic medical record.

33. A robot system, comprising:
a broadband network;
a mobile platform with a camera that captures an image, said mobile platform being coupled to said broadband network; and,
a remote station that is coupled to said mobile platform through said broadband network, said remote station includes a monitor that displays the image, said monitor can also display an electronic medical record.

34. The system of claim 33, wherein said monitor simultaneously displays the image and the electronic medical record.

35. The system of claim 33, wherein the electronic medical record is stored within said mobile platform.

36. The system of claim 33, further comprising a server that is coupled to said remote station through said broadband network and stores the electronic medical record.

37. A method for viewing medical data, comprising:
moving a mobile platform;
capturing an image with a camera of the mobile platform;
transmitting the image to a remote station;
displaying the image on a monitor of the remote station; and,
displaying an electronic medical record on the monitor.

38. The method of claim 37, wherein the electronic medical record is displayed simultaneously with the image.

39. The method of claim 37, further comprising storing the electronic medical record in the mobile robot.

40. The method of claim 37, further comprising transmitting the electronic medical record from a server to the remote station.

41. A display user interface that is displayed on a monitor of a remote station that is coupled to a mobile platform, the mobile platform having a camera that captures an image, comprising:
a field that displays the image and the electronic medical record.

42. The user interface of claim 41, wherein said field simultaneously displays the image and the electronic medical record.

43. The user interface of claim 41, further comprising a button that can selected to view either the image or the electronic medical record.
